(19) United States
(12) Patent Application Publication
(10) Pub. No.: US 2026/0294373 A1
(43) Pub. Date: Oct. 1, 2026

(54) TRUE LUMEN DETECTION AND NAVIGATION USING IMAGING

(71) Applicant: Medtronic Vascular, Inc., Santa Rosa, CA (US)

(72) Inventor: Stephen G. Nash, Athlone (IE)

(21) Appl. No.: 19/490,246

(22) PCT Filed: Jun. 13, 2024
(86) PCT No.: PCT/US2024/033830

§ 371 Date: Dec. 4, 2025

Related U.S. Application Data
(60) Provisional application No. 63/508,133, filed on Jun. 14, 2023.

(51) Int. Cl. A61B 6/50; A61B 6/12; A61B 6/46

(52) U.S. Cl. CPC A61B 6/504; A61B 6/12; A61B 6/461

(57) ABSTRACT

Example medical systems and techniques are disclosed. An example medical system includes memory and processing circuitry. The processing circuitry is configured to obtain angiography data. The processing circuitry is configured to determine, based on the angiography data, a location of a true lumen of a cardiac vessel. The processing circuitry is configured to determine a location of a distal portion of a medical instrument. The processing circuitry is configured to determine, based on the location of the true lumen and the location of the distal portion of the medical instrument, a navigation path for the distal portion of the medical instrument to the true lumen. The processing circuitry is configured to output a representation of the navigation path for display.

This application claims the benefit of U.S. Provisional Application No. 63/508,133, filed Jun. 14, 2023, the entire content of which is hereby incorporated by reference.

### TECHNICAL FIELD

This disclosure relates to the imaging such as imaging used during a medical procedure.

### BACKGROUND

During a medical procedure, a clinician may use an imaging system to be able to visualize internal anatomy of a patient. Such an imaging system may display anatomy, medical instruments, or the like, and may be used to diagnose a patient condition or assist in guiding a clinician in navigating a device inside a patient, such as moving a medical instrument to an intended location inside the patient. Imaging systems may use sensors to capture image data which may be displayed during the medical procedure. Imaging systems include angiography systems, computed tomography (CT) scan systems (including coronary computed tomography angiography (CCTA) systems), fluoroscopic systems (e.g., isocentric C-arm fluoroscopic systems), intravascular ultrasound (IVUS) systems, other ultrasound imaging systems, optical coherence tomography (OCT) fractional flow reserve (FFR) systems, magnetic resonance imaging (MRI) systems, positron emission tomography (PET) systems, as well as other imaging systems.

A chronic total occlusion (CTO) is a medical condition where there is a complete (e.g., total) or nearly complete blockage of a coronary artery. Medical procedures may be used to treat a CTO, such as a percutaneous coronary intervention (PCI), which may attempt to restore blood flow that is occluded by the CTO.

### SUMMARY

During a CTO medical procedure, such as a PCI, it is sometimes desirable to enter a sub intimal space of the occluded vessel to cross the CTO. For example, a clinician may use a sub intimal technique to create a neo-lumen between the intimal and adventitial layers of an arterial wall. In this manner, the clinician may navigate a medical instrument, such as a guidewire, through the neo-lumen to cross the CTO which may reside in a “true” lumen.

However, once the clinician has navigated a portion of the medical instrument into the sub intimal space of a vessel, it may be challenging to determine where the true lumen may be, to navigate the portion of medical instrument back into that true lumen, and to determine that the portion of the medical instrument is actually back in the true lumen. For example, an invasive IVUS catheter may be required to determine that the portion of the medical instrument is back in the true lumen. This may be true because the CTO may block dye used during an angiography from flowing or penetrating to a portion of the vessel that is distal to the CTO. As such, the angiography data may not include enough data to accurately show the location of the true lumen distal of the CTO when displayed to a clinician.

This disclosure describes techniques for navigating to and determining that a distal portion of a medical instrument is back in a true lumen. In some examples, the techniques of this disclosure may include the use of an artificial intelligence or machine learning model to determine or verify that a distal portion of a medical instrument is in a true lumen based on live angiography images.

In one example, the disclosure describes a medical system comprising: memory configured to store angiography data of a patient; and processing circuitry communicatively coupled to the memory, the processing circuitry being configured to: obtain the angiography data; determine, based on the angiography data, a location of a true lumen of a cardiac vessel; determine a location of a distal portion of a medical instrument; determine, based on the location of the true lumen and the location of the distal portion of the medical instrument, a navigation path for the distal portion of the medical instrument to the true lumen; and output a representation of the navigation path for display.

In another example, the disclosure describes a method comprising: obtaining, by processing circuitry, angiography data of a patient; determining, by the processing circuitry and based on the angiography data, a location of a true lumen of a cardiac vessel; determining, by the processing circuitry, a location of a distal portion of a medical instrument; determining, by the processing circuitry and based on the location of the true lumen and the location of the distal portion of the medical instrument, a navigation path for the distal portion of the medical instrument to the true lumen; and outputting, by the processing circuitry, a representation of the navigation path for display.

In yet another example, the disclosure describes a non-transitory computer readable medium comprising instructions, which, when executed, cause processing circuitry to: obtain angiography data of a patient; determine, based on the angiography data, a location of a true lumen of a cardiac vessel; determine a location of a distal portion of a medical instrument; determine, based on the location of the true lumen and the location of the distal portion of the medical instrument, a navigation path for the distal portion of the medical instrument to the true lumen; and output a representation of the navigation path for display.

These and other aspects of the present disclosure will be apparent from the detailed description below. In no event, however, should the above summaries be construed as limitations on the claimed subject matter, which subject matter is defined solely by the attached claims.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a schematic perspective view of one example of a system for guiding navigation of a medical instrument to a true lumen and/or verifying that a distal portion of the medical instrument is in the true lumen according to one or more aspects of this disclosure.

FIG. 2 is a schematic view of one example of a computing system of the system of FIG. 1.

FIG. 3 is a conceptual diagram illustrating an example culprit vessel according to one or more aspects of this disclosure.

FIG. 4 is a conceptual diagram illustrating an example culprit vessel with a medical instrument being navigated through a sub intimal space according to one or more aspects of this disclosure.

FIG. 5 is a conceptual diagram illustrating an example culprit vessel with a medical instrument and a navigation path according to one or more aspects of this disclosure.

FIG. 6 is a flow diagram illustrating example machine learning model verification techniques according to one or more aspects of this disclosure.

FIG. 7 is a conceptual diagram illustrating an example machine learning model according to one or more aspects of this disclosure.

FIG. 8 is a conceptual diagram illustrating an example training process for a machine learning model according to one or more aspects of this disclosure.

FIG. 9 is a conceptual diagram illustrating another example training process for a machine learning model according to one or more aspects of this disclosure.

### DETAILED DESCRIPTION

As discussed above, during a CTO medical procedure, it may be desirable to navigate a distal portion of a medical instrument to enter the sub intimal space of the occluded vessel to cross a CTO. However, once the clinician has navigated the distal portion of the medical instrument into the sub intimal space, it may be challenging to determine where the true lumen may be, to navigate the distal portion of the medical instrument back into that true lumen, and to determine that the medical instrument is actually back in the true lumen.

As such it may be desirable to provide techniques, such as the techniques of this disclosure, for navigating a distal portion of a medical instrument to, and determining that a distal portion of a medical instrument is back in, a true lumen. In some examples, the techniques of this disclosure may include the use of an artificial intelligence or machine learning model (either of which may be referred to hereinafter as a machine learning model) to determine or verify that a distal portion of a medical instrument is in a true lumen based on live angiography images.

During a CTO procedure, a clinician may advance a medical instrument, such as a hydrophilic guidewire with a hydrophilic support catheter in the true lumen of a culprit vessel toward the CTO. The clinician may direct the guidewire tip toward the arterial wall at or near the site of the CTO in order to penetrate into the sub intimal space of the culprit vessel wall. Once the guidewire tip has entered the sub intimal space, there is generally little resistance, and the clinician may advance the distal portion of the medical instrument past the CTO (e.g., cross the occlusion).

In some examples, the clinician may use a wire loop to dissect the sub intimal space between the neo-lumen and the true lumen to return the distal portion of the medical instrument to the true lumen. For example, the clinician may use a relatively narrow wire loop to re-enter the true lumen. However, it may be relatively difficult for the clinician to know the direction of the true lumen or when the true lumen has been re-entered, for example, when IVUS is not used.

The techniques of this disclosure may utilize image processing to analyze angiography data from a diagnostic angiograph of the culprit vessel. The image processing may identify the location of the CTO within the vessel and characterize the occlusion accordingly. During the PCI procedure to treat the CTO, the system, executing the machine learning model and/or artificial intelligence (AI) model, may utilize the live angiography feed to identify the location of a medical instrument, such as a guide wire relative to the true lumen. The system may confirm that a distal portion of the medical instrument is in the true lumen when that is the case or inform the clinician that the distal portion of the medical instrument is not in the true lumen, and, in some examples, provide navigation guidance towards the true lumen.

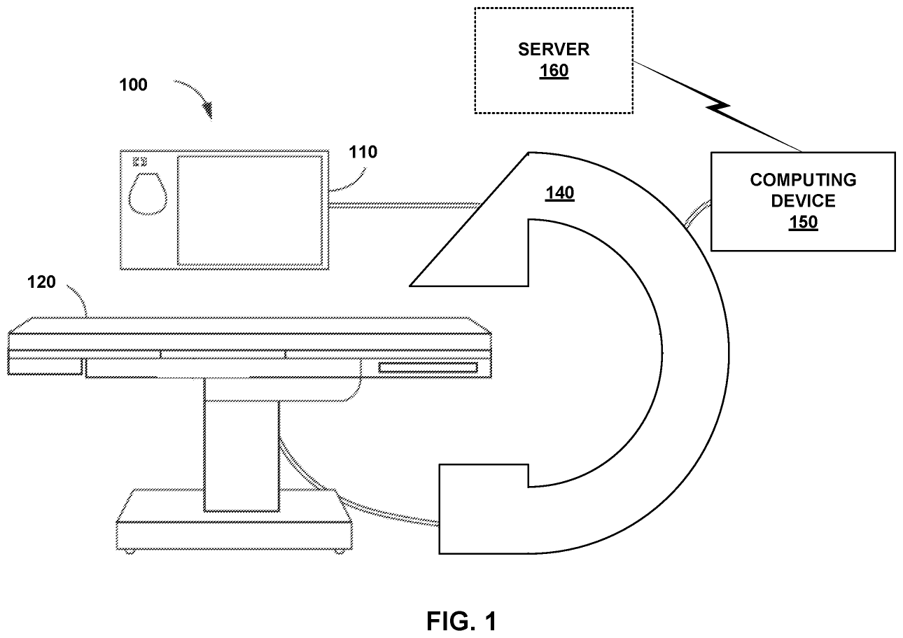

FIG. 1 is a schematic perspective view of one example of a system for guiding navigation of a medical instrument to a true lumen and/or verifying that a distal portion of the medical instrument is in the true lumen according to one or more aspects of this disclosure. System 100 includes a display device 110, a table 120, an imager 140, and a computing device 150. System 100 may be an example of a system for use in an emergency room or a Catheterization laboratory (Cath lab). In some examples, system 100 may include other devices, not shown for simplicity purposes. In some examples, system 100 may also include server 160, which may be co-located with the other devices of system 100 or may be located elsewhere. System 100 may be used during a medical procedure, such as an interventional medical procedure like a PCI and/or a diagnostic medical procedure. During such a medical procedure a clinician may address a CTO, such as by attempting to cross the CTO through a neo-lumen between the intimal and adventitial layers of an arterial wall.

Computing device 150 may include, for example, an off-the-shelf device such as a laptop computer, desktop computer, tablet computer, smart phone, or other similar device or may include a specific purpose device. Computing device 150 may perform various control functions with respect to imager 140. In some examples, computing device 150 may include a guidance workstation. Computing device 150 may control the operation of imager 140 and receive the output of imager 140 and may receive angiography data from imager 140. Computing device 150 may execute the machine learning algorithm and determine the true lumen, determine a navigation path of at least a distal portion of a medical instrument, and/or determine whether the distal portion of the medical instrument is in the true lumen.

Display device 110 may be configured to output instructions, images, and messages relating to the medical procedure(s). For example, display device 110 may display angiography data obtained through imager 140 and/or a representation of the navigation path. Table 120 may be, for example, an operating table or other table suitable for use during a medical procedure.

In the example of FIG. 1, imager 140, such as an angiography imager (or other imaging device), may be used to image relevant portions of the patient's anatomy during a medical procedure to visualize the anatomy, characteristics and locations of lesions or other issues inside the patient's body through the generation of imaging data. While described herein primarily as an angiography imager, imager 140 may be any type of imaging device, such as an angiography device, a fluoroscopy device, a CT device, a CCTA device, an IVUS device, an OCT-FFR device, an MRI device, a PET device, an ultrasound device, or the like. In some examples, imager 140 may represent more than one imaging device, such as a plurality of any of the aforementioned devices.

Imager 140 may image a region of interest in the patient's body. The particular region of interest may be dependent on anatomy, the medical procedure, patient symptoms, and/or the like. For example, when performing a cardiac medical procedure, a portion of the vasculature and/or the heart may be within the region of interest.

Computing device 150 may be communicatively coupled to imager 140, display device 110 and/or server 160, for example, by wired, optical, or wireless communications. Server 160 may be a hospital server which may or may not be located in an emergency room or Cath lab of a hospital, a cloud-based server, or the like. Server 160 may be configured to store patient imaging data (such as angiography data), electronic healthcare or medical records, or the like. In some examples, server 160 may be configured to execute the machine learning model(s) and/or perform one or more of, or a portion of one or more of, the determinations associated therewith.

Any of, or any combination of, computing device 150, imager 140, and/or server 160 may include one or more machine learning model(s). For example, computing device 150, imager 140, and/or server 160 may obtain angiography data, e.g., via imager 140. Computing device 150, imager 140, and/or server 160 may determine, based on the angiography data, a location of a true lumen of a cardiac vessel. For example, computing device 150, imager 140, and/or server 160 may determine the location of the true lumen by executing a machine learning model. Computing device 150, imager 140, and/or server 160 may determine a location of a distal portion of a medical instrument. Computing device 150, imager 140, and/or server 160 may determine, based on the location of the true lumen and the location of the distal portion of the medical instrument, a navigation path to the location of the true lumen. For example, computing device 150, imager 140, and/or server 160 may determine the navigation path by executing a machine learning model. Computing device 150, imager 140, and/or server 160 may output a representation of the navigation path for display, for example, to display device 110.

By determining and outputting a representation of the navigation path, system 100 may assist clinicians in more effectively guiding treatment of a lesion, such as a CTO lesion, during a PCI medical procedure. As such, the techniques of this disclosure may improve patient outcomes and/or medical facility efficiency.

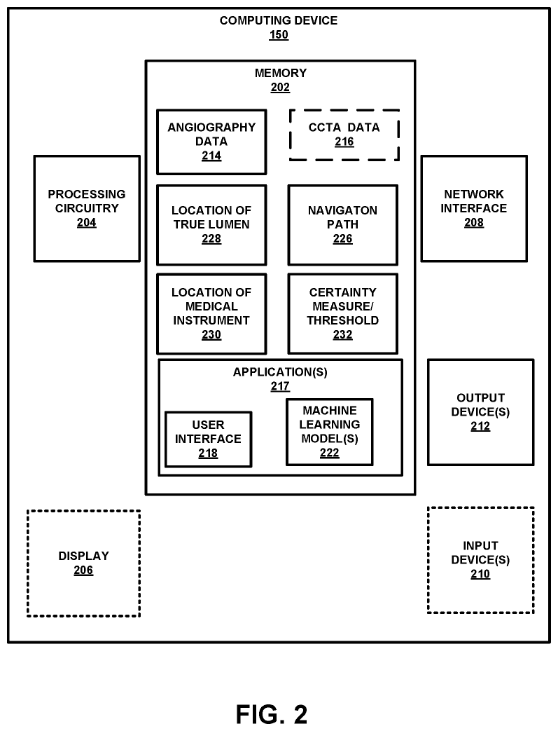

FIG. 2 is a schematic view of one example of a computing device 150 of system 10 of FIG. 1. Computing device 150 may include a workstation, a desktop computer, a laptop computer, a smart phone, a tablet, a dedicated computing device, or any other computing device capable of performing the techniques of this disclosure.

Computing device 150 may be configured to perform processing, control and other functions associated with imager 140. As shown in FIG. 2, computing device 150 may represent multiple instances of computing devices, each of which may be associated with imager 140. Computing device 150 may include, for example, a memory 202, processing circuitry 204, a display 206, a network interface 208, input device(s) 210, and/or output device(s) 212, each of which may represent any of multiple instances of such a device within the computing system, for ease of description.

While processing circuitry 204 appears in computing device 150 in FIG. 2, in some examples, features attributed to processing circuitry 204 may be performed by processing circuitry of any of computing device 150, imager 140, or server 160, or combinations thereof. In some examples, one or more processors associated with processing circuitry 204 in computing system may be distributed and shared across any combination of computing device 150, imager 140, and server 160. Computing device 150 may be used to perform any of the techniques described in this disclosure, and may form all or part of devices or systems configured to perform such techniques, alone or in conjunction with other components, such as components of computing device 150, imager 140, server 160, or a system including any or all of such systems/devices.

Memory 202 of computing device 150 includes any non-transitory computer-readable storage media for storing data or software that is executable by processing circuitry 204 and that controls the operation of computing device 150 and/or imager 140, as applicable. In one or more examples, memory 202 may include one or more solid-state storage devices such as flash memory chips. In one or more examples, memory 202 may include one or more mass storage devices connected to the processing circuitry 204 through a mass storage controller (not shown) and a communications bus (not shown).

Although the description of computer-readable media herein refers to a solid-state storage, it should be appreciated by those skilled in the art that computer-readable storage media may be any available media that may be accessed by the processing circuitry 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by computing device 150. In one or more examples, computer-readable storage media may be stored in the cloud or remote storage and accessed using any suitable technique or techniques through at least one of a wired or wireless connection.

Memory 202 may store angiography data 214, CCTA data 216, location of true lumen 228, location of medical instrument 230, navigation path 226, and/or certainty measure/threshold 232. Angiography data 214 may include a plurality of images obtained, for example, from imager 140 during the medical procedure. In some examples, angiography data 214 may also include images obtained during a prior diagnostic angiography medical procedure. While the medical procedure is proceeding in time, additional angiography data may be obtained from imager 140 and stored in angiography data 214. Such angiography data may be displayed via display 206 and/or display device 110 and may be used by a clinician when navigating a medical instrument through anatomy of a patient.

Angiography data 214 may be generated by imager 140 of anatomy of the patient and obtained by computing device 150 via network interface 208 which may be communicatively coupled to imager 140. In some examples, imager 140 may generate other types of imaging data, such as when imager 140 represents more than one imaging device. For example, imager 140 may generate IVUS data, which may be used to train machine learning model(s) 222, and/or CCTA data 216. CCTA data 216 may include CCTA data of a current patient taken during a diagnostic procedure and may include lesion geometry, length, morphology, tissue composition, and/or the like.

For example, angiography data 214 and/or CCTA data 216 may be captured by imager 140 (FIG. 1). Processing circuitry 204 may obtain angiography data 214 and/or CCTA data 216 from imager 140 and store angiography data 214 and/or CCTA data 216 in memory 202. Processing circuitry 204 may execute user interface 218 so as to cause display 206 (and/or display device 110 of FIG. 1) to present user interface 218 to one or more clinicians performing the medical procedure. User interface 218 may display angiography data 214 and/or CCTA data 216.

Location of true lumen 228 may include a location of a distal true lumen, distal to a lesion, such as a CTO lesion. Location of true lumen 228 may be determined by processing circuitry 204 executing machine learning model(s) 222. Location of medical instrument 230 may include a location of a distal portion of a medical instrument. Location of medical instrument 230 may be determined by processing circuitry 204 using any instrument tracking technology, such as electromagnetic tracking, computer vision (which may be implemented via machine learning model(s) 222), or the like.

Certainty measure/threshold 232 may include a measure of a level of confidence in a determination of location of true lumen 228. Certainty measure/threshold 232 may also include a threshold against which processing circuitry 204 may compare the certainty measure. These are described later hereinafter.

Memory 202 may also store one or more machine learning model(s) 222 and user interface 218. Machine learning model(s) 222 may be configured to, when executed by processing circuitry 204, determine location of true lumen 228 of a cardiac vessel, determine navigation path 226 to the location of the true lumen, and/or determine whether a distal portion of a medical instrument is located in the true lumen. By determining the location of the true lumen, determining how to navigate a distal portion of a medical instrument to the true lumen, and verifying whether the distal portion of the medical instrument is in the true lumen, the techniques of this disclosure may provide greater confidence in clinicians in crossing a lesion, such as a CTO, when using a neo-lumen.

In some examples, angiography data 214 includes live angiography data, angiography data captured before the current medical procedure, during the current medical procedure, after a medical procedure, or the like. In some examples, processing circuitry 204 may use angiography data 214 to build a 3D model of the anatomy of the patient in at least an area proximate to the lesion, to provide a clinician with a visualization of anatomy distal of the occlusion. In some examples, processing circuitry 204 may augment angiography data 214 with CCTA data 216 to provide a better picture of vessels, e.g., morphology, composition, plaque, calcium, etc.

Processing circuitry 204 may execute machine learning model(s) 222, which may include a neural network or other type of machine learning model to identify when the distal portion of the medical instrument is in the true lumen, when the distal portion of the medical instrument is not in the true lumen, and how to navigate the distal portion of the medical instrument to the true lumen. Machine learning model(s) 222 may be trained based on angiography data of medical procedures, such as diagnostic and/or interventional medical procedures, cine imaging data, annotations regarding which medical instruments are used during sub intimal CTO PCIs, IVUS data, CCTA data, or the like. For example, during a medical procedure, an IVUS sensor may actually be located within the true lumen of a culprit vessel and the IVUS data generated by the IVUS sensor may be used to verify whether a distal portion of a medical instrument is within the true lumen. As such, IVUS data and corresponding angiography data from a same medical procedure may be used to determine characteristics in the angiography data which are indicative of the distal portion of a medical instrument being in or out of the true lumen. By training machine learning model(s) 222 using both the angiography data and the IVUS data, machine learning model(s) 222 may be trained to identify the true lumen using angiography data 214 alone, thus reducing or eliminating the need for IVUS during CTO procedures.

In some examples, machine learning model(s) 222 may be further trained with, or processing circuitry 204 may otherwise employ, pre-procedure CCTA data 216. For example, machine learning model(s) 222 may use pre-procedure CCTA data 216 of the current patient and angiography data 214 to further enhance the accuracy of the true lumen detection and navigation. For example, CCTA data 216 may include information regarding the lesion, such as lesion geometry, length, morphology, tissue composition, and/or the like. By using CCTA data 216, machine learning model(s) 222 may learn a location of not only the proximal end of the lesion, but also of the distal end of the lesion, thereby facilitating the navigation of the distal portion of a medical instrument through the sub intimal space passed the lesion and back into the true lumen of the culprit vessel distal to the distal end of the lesion.

For example, processing circuitry 204 executing machine learning model(s) 222 may determine navigation path 226 for the distal portion of a medical instrument. Navigation path 226 may include a recommended angle or direction of approach, and/or distance towards the true lumen when the distal portion of the medical instrument is not in the true lumen, but has crossed the lesion. Processing circuitry 204 may output the recommended angle, direction of approach, and/or distance to display 206 and/or display device 110, for use by the clinician, should the clinician decide to follow the recommendation.

Processing circuitry 204 executing machine learning model(s) 222 may determine location of true lumen 228 of the occluded vessel and may output a representation of location of true lumen 228 to a display, such as display 206 and/or display device 110, so as to enable a clinician to readily identify the true lumen. Processing circuitry 204 may output the representation of the location of the true lumen during the entire medical procedure or during a portion of the medical procedure. For example, processing circuitry 204 may be configured to output the representation of the location of the true lumen once the distal portion of a medical device deviates from the true lumen, such as enters a neo lumen.

In some examples, processing circuitry 204 may determine a certainty measure of certainty measure/threshold 232 associated with the determination of location of true lumen 228. The certainty measure may be indicative of a level of confidence that location of true lumen 228 is accurate. For example, processing circuitry 204 may determine whether the certainty measure satisfies a threshold, also of certainty measure/threshold 232. For the purposes of this disclosure, satisfying the threshold should be understood to mean that the level of confidence is more confident than the threshold or equal to or more confident than the threshold, whether that amounts to the certainty measure being greater than or equal to, greater than, less than or equal to, or less than the threshold. If the certainty measure does not satisfy the threshold (e.g., the level of confidence is not sufficiently high as to the location of the true lumen), processing circuitry 204 may prompt a clinician to acquire additional angiography data from a different angle so as to add additional data and improve (or not) the level of confidence in the location of the true lumen. Once the certainty measure satisfies the threshold (e.g., the level of confidence is sufficiently high as to the location of the true lumen), processing circuitry 204 may determine navigation path 226 and output a representation thereof for display to display 206 and/or display device 110.

In some examples, machine learning model(s) 222 may be built utilizing the following ground truth data. For angiography data of a CTO with no visual data of the distal true lumen: angiography data of CTO with no visual of the distal true lumen from previous medical procedure(s); CCTA data from previous medical procedure(s) (if available); final successful case angiography data; IVUS data, OCT data and/or other intravascular imaging data (if available); electrocardiogram (ECG) data (if available); and/or the like. For angiography data of CTO with visual of the distal true lumen through collateral device(s): angiography data of CTO with visual of the distal true lumen from previous medical procedure(s) through collateral device(s); CCTA data from previous medical procedure(s) (if available); final successful case angiography data; IVUS data, OCT data and/or other intravascular imaging data (if available); ECG data (if available); and/or the like. The ground truth data may be utilized to train an algorithm to predict the location of the true lumen.

Processing circuitry 204 may be implemented by one or more processors, which may include any number of fixed-function circuits, programmable circuits, or a combination thereof. In various examples, control of any function by processing circuitry 204 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 204 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Display 206 may be touch sensitive or voice activated, enabling display 206 to serve as both an input and output device. Alternatively, a keyboard (not shown), mouse (not shown), or other data input devices (e.g., input device(s) 210) may be employed.

Network interface 208 may be adapted to connect to a network such as a local area network (LAN) that includes a wired network or a wireless network, a wide area network (WAN), a wireless mobile network, a Bluetooth network, or the internet. For example, computing device 150 may obtain angiography data 214 from imager 140 during a medical procedure. Computing device 150 may receive updates to its software, for example, application(s) 217, via network interface 208. Computing device 150 may also display notifications on display 206 that a software update is available.

Input device(s) 210 may include any device that enables a user to interact with computing device 150, such as, for example, a mouse, keyboard, foot pedal, touch screen, augmented-reality input device receiving inputs such as hand gestures or body movements, or voice interface.

Output device(s) 212 may include any connectivity port or bus, such as, for example, parallel ports, serial ports, universal serial busses (USB), or any other similar connectivity port known to those skilled in the art.

Application(s) 217 may be one or more software programs stored in memory 202 and executed by processing circuitry 204 of computing device 150. Processing circuitry 204 may execute user interface 218, which may display angiography data 214, location of true lumen 228, and/or navigation path 226 for the medical instrument to the true lumen on display 206 and/or display device 110. A clinician may use the displayed data or navigation path 226 to guide and advance a distal portion of a medical instrument from within a neo lumen into a true lumen when crossing a lesion.

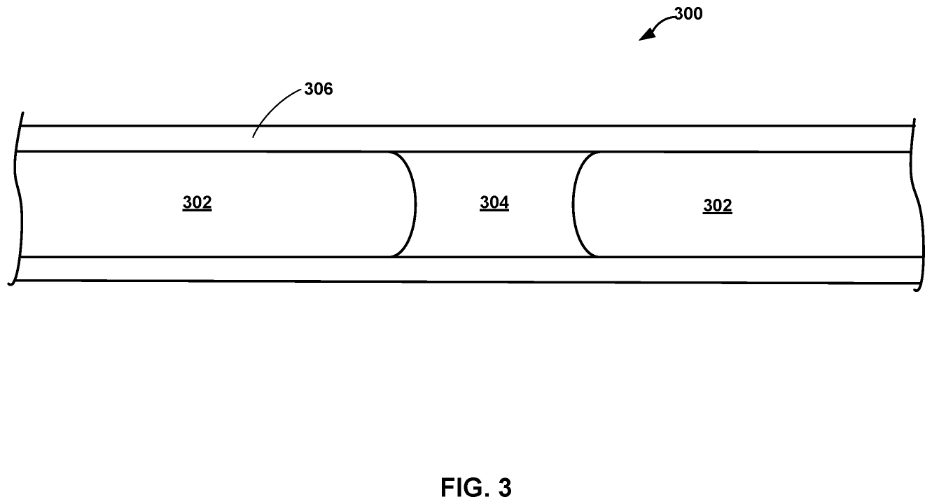

FIG. 3 is a conceptual diagram illustrating an example culprit vessel according to one or more aspects of this disclosure. Culprit vessel 300 may include a true lumen 302. True lumen may be a portion of culprit vessel 300 through which blood would normally flow if culprit vessel were not occluded. Culprit vessel 300 may include lesion 304 which may be a CTO. Culprit vessel 300 may also include sub intimal space 306. Sub intimal space 306 may be a space between true lumen 302 and an exterior of culprit vessel 300. In some examples, when angiography data 214 is displayed on display 206 and/or display device 110, processing circuitry 204 may use a different color on the display to represent true lumen 302 than to represent sub intimal space 306. For example, processing circuitry 204 may colorize or overlay colored representations of true lumen 302 and sub intimal space 306 on angiography data 214 on a display so as to enable a clinician to readily distinguish true lumen 302 from sub intimal space 306.

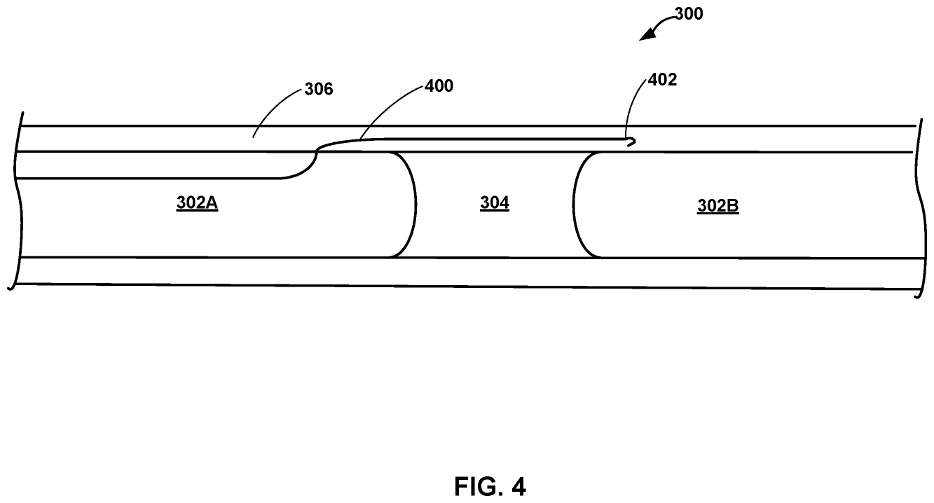

FIG. 4 is a conceptual diagram illustrating an example culprit vessel with a medical instrument being navigated through a sub intimal space according to one or more aspects of this disclosure. Lesion 304 may block the flow of blood through true lumen 302 (FIG. 3) from a proximal true lumen 302A to a distal true lumen 302B. As such, a clinician may desire to cross lesion 304 to restore or improve blood flow through culprit vessel 300. As such, a clinician may navigate a distal portion 402 of medical instrument 400 through proximal true lumen 302A towards lesion 304. When distal portion 402 of medical instrument 400 nears lesion 304, the clinician may guide distal portion 402 medical instrument into sub intimal space 306 in an attempt to cross lesion 304. Because lesion 304 blocks the flow of blood through true lumen 302 of culprit vessel 300, live angiography data of culprit vessel may not show the portion of true lumen 302 that is distal to lesion 304. As such it may be desirable for processing circuitry 204 to determine a location of distal true lumen 302B which is distal to lesion 304 and/or for processing circuitry 204 to determine a navigation path for distal portion 402 of medical instrument 400 from sub intimal space 306 to distal true lumen 302.

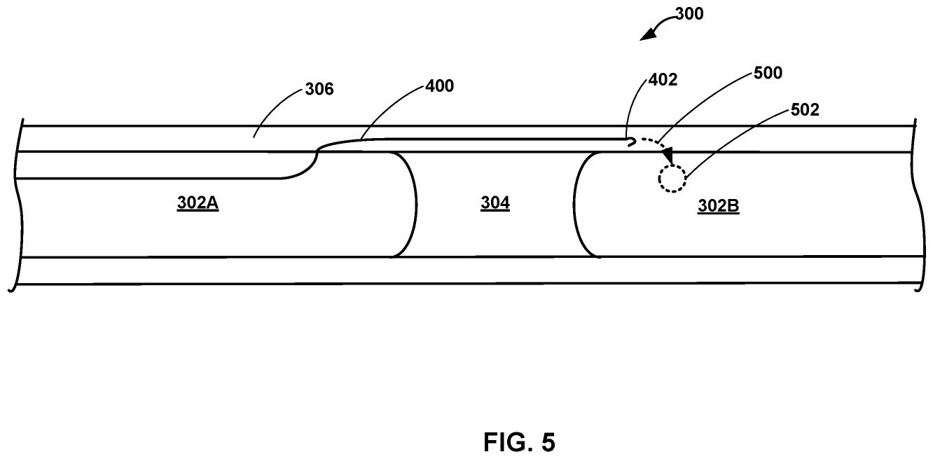

FIG. 5 is a conceptual diagram illustrating an example culprit vessel with a medical instrument and a navigation path according to one or more aspects of this disclosure. Processing circuitry 204, for example, after distal portion 402 of medical instrument 400 enters sub intimal space 306, a location of distal true lumen 302B (e.g., a portion of true lumen 302 distal of lesion 304). Processing circuitry 204 may also determine a navigation path 500 for distal portion 402 of medical instrument 400 from sub intimal space 306 to distal true lumen 302B. Processing circuitry 204 may output a representation of true lumen 302 (FIG. 3) including distal true lumen 302B and/or navigation path 500 to display 206 and/or display device 110 for display to a clinician. In this manner, a clinician may use the displayed representation of true lumen 302 and/or displayed representation of navigation path 500 (which may be an example of navigation path 226 of FIG. 2) to guide medical instrument 400 such that a distal portion 402 of medical instrument 400 back into true lumen 302 distal to lesion 304 and cross lesion 304. Navigation path 500 may include an angle or direction of approach and/or a distance. In some examples, navigation path 500 may include an indication to a clinician (e.g., via display 206, display device 110, and/or output device(s) 212, when distal portion 402 passes distally of lesion 304 and prompt the clinician to start navigating towards true lumen 302, e.g., distal true lumen 302B, for example along navigation path 500.

In some examples, processing circuitry 204 may determine that distal portion 402 of medical instrument 400 has re-entered true lumen 302 (e.g., entered distal true lumen 302B) and may output an indication 502, for example, for display on display 206 and/or display device 110. Indication 502 is represented in FIG. 5 by a circle, but may include any visual or auditory indication to a clinician that distal portion 402 of medical instrument 400 has re-entered true lumen 302.

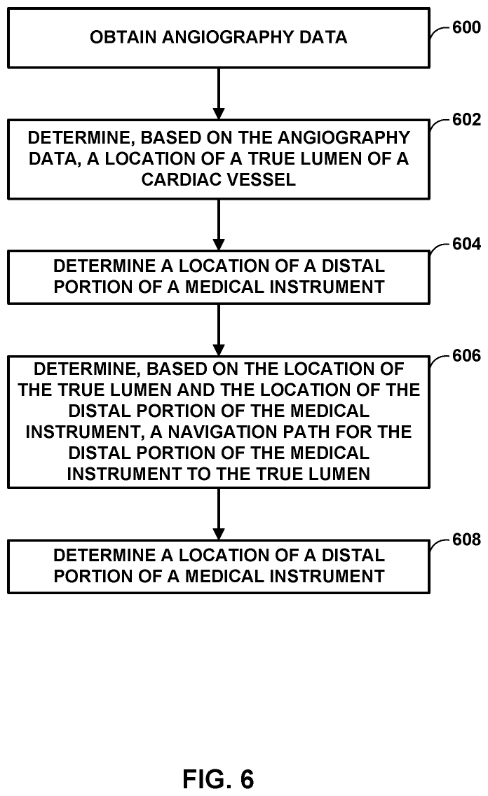

FIG. 6 is a flow diagram of example techniques for determining a recommended treatment strategy according to one or more aspects of this disclosure. The techniques of FIG. 6 are described below with respect to processing circuitry 204, but such techniques may be performed by any of, or any combination of, processing circuitry of devices depicted in FIG. 1 or capable of performing such techniques.

Processing circuitry 204 may obtain the angiography data (600). For example, processing circuitry 204 may read angiography data 214 from memory 202 or receive angiography data 214 from imager 140 via, for example, network interface 208.

Processing circuitry 204 may determine, based on the angiography data, a location of a true lumen of a cardiac vessel (602). For example, processing circuitry 204 may execute one or more machine learning model(s) 222 to determine location of true lumen 228 based on angiography data 214.

Processing circuitry 204 may determine a location of at least a distal portion of a medical instrument (604). For example, processing circuitry 204 may determine location of the distal portion of the medical instrument (e.g., of location of medical instrument 230) by executing one or more machine learning model(s) 222 based on angiography data 214, by using an electromagnetic tracking technique, computer vision, or the like.

Processing circuitry 204 may determine, based on the location of the true lumen and the location of at least the distal portion of the medical instrument, a navigation path for the of the medical instrument to the true lumen (606). For example, processing circuitry 204 may determine navigation path 226 by which a clinician may navigate distal portion 402 of medical instrument 400 from the location of the distal portion of the medical instrument into the true lumen (e.g., distal true lumen 302B of FIGS. 4-5).

Processing circuitry 204 may output a representation of the navigation path for display (608). For example, processing circuitry 204 may output to display 206 and/or display device 110, a representation of navigation path 226 or how distal portion 402 of medical device 400 should be navigated to re-enter the true lumen (e.g., distal true lumen 302B). Such a representation may be a graphical representation to be displayed in a particular color, shading, or by one or more lines which may distinguish navigation path 226 from angiography data 214 being displayed on display 206 and/or display device 110. Processing circuitry 204 may overlay navigation path 226 on the displayed angiography data 214, thereby facilitating a clinician navigating distal portion 402 of medical instrument 400 into the true lumen (e.g., distal true lumen 302B).

In some examples, processing circuitry 204 is further configured to determine, based on angiography data 214, a location of a lesion 304 (FIGS. 3-5), wherein processing circuitry 204 is configured to determine navigation path 226 further based on the location of the lesion 304. In this manner, a clinician may avoid trying to cause distal portion 402 of medical instrument 400 to re-enter true lumen 302 either before or along the length of the lesion. In some examples, the lesion is a CTO.

In some examples, as part of at least one of determining location of true lumen 228 or determining navigation path 226, processing circuitry 204 is configured to execute machine learning model(s) 222. In some examples, machine learning model(s) is trained on at least one of angiography data of CTO medical procedures, cine imaging data, annotations associated with medical instruments used during sub intimal CTO medical procedures, IVUS data, or CCTA data.

In some examples, as part of determining navigation path 226, processing circuitry 204 is configured to determine a first certainty measure (e.g., of certainty measure/threshold 232) associated with the determination of location of true lumen 228. In such examples, processing circuitry 204 is configured to determine whether the first certainty measure satisfies a threshold (e.g., of certainty measure/threshold 232). In such examples, based on the first certainty measure not satisfying the threshold, processing circuitry 204 is configured to output a request to acquire additional angiography data at a different angiography angle than a current angiography angle. In some examples, processing circuitry 204 is further configured to acquire the additional angiograph data (e.g., of angiography data 214). In some examples, processing circuitry 204 is further configured to determine, based on the angiography data and the additional angiography data, a second estimated location of the true lumen. In such examples, processing circuitry 204 is configured to determine a second certainty measure associated with the determination of the second estimated location of the true lumen. In such examples, processing circuitry 204 is configured to determine whether the second certainty measure satisfies the threshold. In some examples, processing circuitry 204 is configured to, based on the second certainty measure satisfying the threshold, determining navigation path 226.

In some examples, the representation of navigation path 226 includes an instruction to navigate distal portion 402 of medical instrument 400 into true lumen 302. In some examples, the representation of navigation path 226 includes a graphical representation of the navigation path.

In some examples, processing circuitry 204 is further configured to obtain updated angiography data. In some examples, processing circuitry 204 is further configured to determine, based on the updated angiography data, an updated location of the true lumen. In some examples, processing circuitry 204 is further configured to determine an updated location of distal portion 402 of medical instrument 400. In some examples, processing circuitry 204 is further configured to determine, based on the updated location of true lumen 302 and the updated location of distal portion 402 of medical instrument 400, whether distal portion 402 of medical instrument 400 is in true lumen 302. In some examples, processing circuitry 204 is configured to, based on distal portion 402 of medical instrument 400 being in true lumen 302, output indication 502. In some examples, processing circuitry 204 is configured to output indication 502 for display.

In some examples, navigation path 226 includes at least one of an angle of approach towards true lumen 302, a direction of navigation towards true lumen 302 or a distance from medical instrument 400 to true lumen 302.

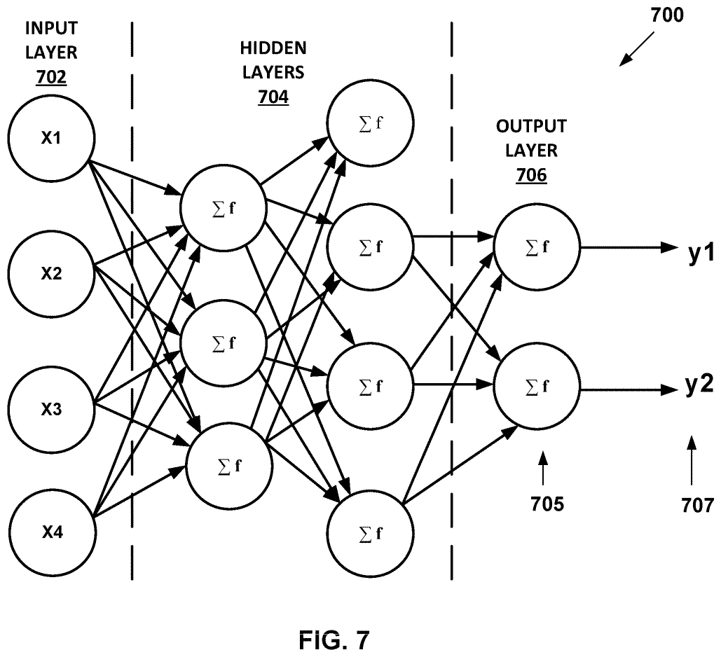

FIG. 7 is a conceptual diagram illustrating an example machine learning model according to one or more aspects of this disclosure. Machine learning model 700 may be an example of machine learning model(s) 222. Machine learning model 700 may be an example of a deep learning model, or deep learning algorithm, trained to determine the location of a true lumen and a navigation path for a medical instrument to the true lumen. One or more of computing device 150 and/or server 160 may train, store, and/or utilize machine learning model 700, but other devices of system 100 may apply inputs to machine learning model 700 in some examples. In some examples, various types of machine learning and deep learning models or algorithms may be utilized. For examples, a convolutional neural network model, e.g., ResNet-18, may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 7, machine learning model 700 may include three types of layers. These three types of layers include input layer 702, hidden layers 704, and output layer 706. Output layer 706 comprises the output from the transfer function 705 of output layer 706. Input layer 702 represents each of the input values X1 through X4 provided to machine learning model 700. In some examples, the input values may include any of the values input into the machine learning model, as described above. For example, the input values may include angiography data 214, as described above. In addition, in some examples input values of machine learning model 700 may include additional data, such as other data that may be collected by or stored in system 100.

Each of the input values for each node in the input layer 702 is provided to each node of a first layer of hidden layers 704. In the example of FIG. 7, hidden layers 704 include two layers, one layer having four nodes and the other layer having three nodes, but fewer or greater number of nodes may be used in other examples. Each input from input layer 702 is multiplied by a weight and then summed at each node of hidden layers 704. During training of machine learning model 700, the weights for each input are adjusted to establish a relationship between angiography data 214, and the location of the true lumen and/or a navigation path for the medical instrument to the true lumen. In some examples, one hidden layer may be incorporated into machine learning model 700, or three or more hidden layers may be incorporated into machine learning model 700, where each layer includes the same or different number of nodes.

The result of each node within hidden layers 704 is applied to the transfer function of output layer 706. The transfer function may be linear or non-linear, depending on the number of layers within machine learning model 700. Example non-linear transfer functions may be a sigmoid function or a rectifier function. The output 707 of the transfer function may be a classification that an area within angiography data 214 is indicative of a true lumen and/or a navigation path to the true lumen.

As shown in the example above, by applying machine learning model 700 to input data such as angiography data 214, processing circuitry 204 is able to determine the location of the true lumen and/or a navigation path for the medical instrument to the true lumen. This may improve the ability of a clinician to navigate the medical instrument through the neo lumen back in the true lumen to cross a CTO.

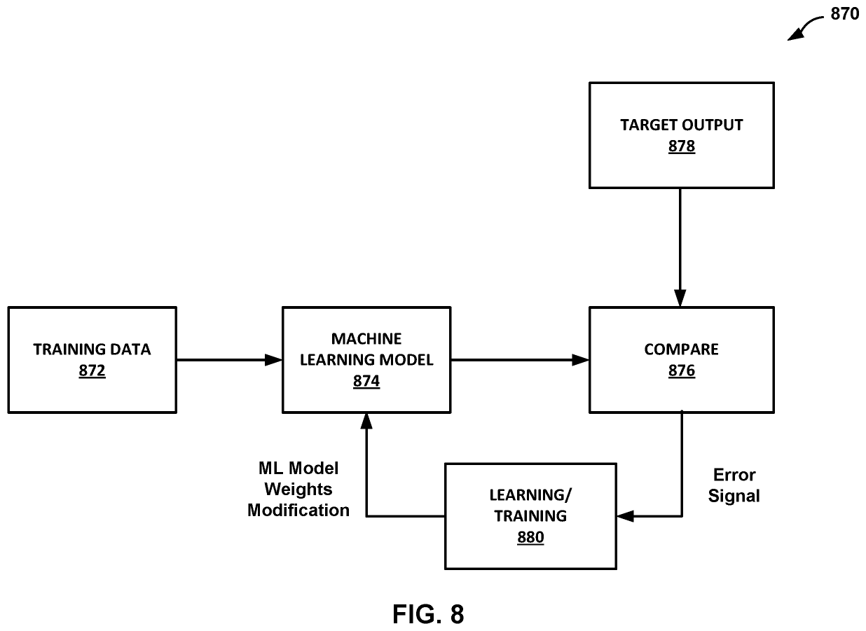

FIG. 8 is a conceptual diagram illustrating an example training process for a machine learning model according to one or more aspects of this disclosure. Process 870 may be used to train machine learning model(s) 222 or machine learning model 700. A machine learning model 874 (which may be an example of machine learning model 700 and/or machine learning model(s) 222) may be implemented using any number of models for supervised and/or reinforcement learning, such as but not limited to, an artificial neural network, a decision tree, naïve Bayes network, support vector machine, or k-nearest neighbor model, convolutional neural network (CNN), recursive neural network (RNN), long short-term memory (LSTM), ensemble network, to name only a few examples.

In some examples, one or more of computing device 150 and/or server 160 initially trains machine learning model 874 based on a corpus of training data 872. Training data 872 may include, for example, angiography data of CTO medical procedures, such as diagnostic and/or interventional medical procedures, cine imaging data, annotations regarding medical instruments used during sub intimal CTO PCIs, IVUS data, CCTA data, or the like. In some examples, training data 872 may include annotations identifying specific medical instruments used during sub intimal CTO PCIs.

In some examples, training data 872 may include images of anatomy of the current patient, images of anatomy of other patients, and/or the like. For example, training data 872 may include data from past medical procedures performed on a plurality of patients having different patient conditions, different prior medical procedures, annotations or tags, other training data mentioned herein, and/or the like.

For example, during some medical procedures and an IVUS imager may actually be at least partially located within the true lumen of the culprit vessel. As such, data from the IVUS imaged may be used to verify that the medical instrument is within the true lumen or not. By training machine learning model 874 using both angiography data and IVUS data, machine learning model 874 may be trained to identify the true lumen using angiography data 214 alone, thus reducing or eliminating the need for IVUS during CTO procedures.

For example, training data 872 may include pre-procedure CCTA data of the current patient. The CCTA may act as a map onto which processing circuitry 204 may overlay live angiography data. For example, processing circuitry 204 may use a relevant CCTA file of the current patient and with angiography data 214 to further enhance the accuracy of the true lumen detection and navigation. In some examples, with the subsequent medical follow up of patients, treatment and outcome data may be used to train a machine learning algorithm of machine learning algorithm(s) 222 to recommend medical treatments, treatment strategies, and/or recommended medical instruments and/or devices.

While training machine learning model 874, processing circuitry of system 100 may compare 876 a prediction or classification with a target output 878. Processing circuitry 204 may utilize an error signal from the comparison to train (learning/training 880) machine learning model 874. Processing circuitry 204 may generate machine learning model weights or other modifications which processing circuitry 204 may use to modify machine learning model 874. For examples, processing circuitry 204 may modify the weights of machine learning model 874 based on the learning/training 880. For example, one or more of computing device 150 and/or server 160, may, for each training instance in training data 872, modify, based on training data 872, the manner in which a location of a true lumen and/or a navigation path for a medical instrument is determined and/or identified.

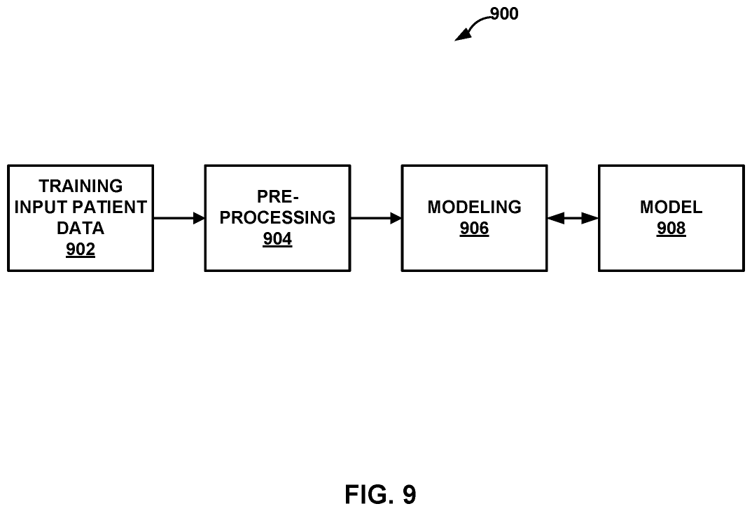

FIG. 9 is a conceptual diagram illustrating another example training process for a machine learning model according to one or more aspects of this disclosure. Process 900 may be used to train machine learning model(s) 222 or machine learning model 700. A machine learning model 908 (which may be an example of machine learning model 700 and/or machine learning model(s) 222) may include a neural network or other type of machine learning model.

Training data 902 may include patient data. In some examples, training data 902 include patient data from approximately 5000 patient cases (or more). Such patient data may include angiography data (e.g., X-ray data) from pre-, peri-, and/or post-CTO PCI procedures. In some examples, training data 902 includes annotated images including entering medical instruments, such as guide wires, catheters, etc., through a cardiac vessel, such as an artery, and through a subintimal space. Training data 902 may include images and/or annotations of the anatomy, such as the vessel, a lesion, dimensions, collateral flow, anatomy and/or other information distal to CTO if visible, other vessels (for example, contra laterals), and/or the like.

Training data 902 may also include CCTA data from procedures prior to the CTO PCIs. In some examples, the CCTA data is related to the CTOs. In some examples, the CCTA data is for the same patients as the angiography data of training data 902. The CCTA data may include images and/or annotations of the anatomy, such as the vessel, a lesion, dimensions, collateral flow, anatomy and/or other information distal to CTO if visible, other vessels (for example, contra laterals), and/or the like.

Training data 902 may also include ECG data, for example, for the same patients as the other training data. Training data 902 may include confirmation of successful true lumen entrance for the medical instrument verified by IVUS data for the same patients as the other training data. Training data 902 may also include images and/or other information relating to various types of medical instruments, such as guide wires, guide catheters, or other medical instruments used in PCIs to treat CTOs. Such images and/or other information relating to the various types of medical instruments may be from the procedures relating to the patient cases for the other training data and/or from other sources.

The input training data 902 may be pre-processed 904, for example, by a neural network. The pre-processed data may be modeled 906 to generate machine learning model 908. For example, modeling 906 may generate a transfer function (Fx) that may be applied to input data to make a prediction regarding the location of the true lumen of a vessel and/or whether a distal portion of the medical instrument is in the true lumen or not. Such input data may include CCTA captured prior to the CTO PCI, angiography data captured prior to the CTO PCI (e.g., diagnostic angiography images), and/or live CTO PCI angiography data, such as when a medical instrument is entering a vessel, the medical instrument in the vessel, subintimal space and/or the true lumen. It should be noted that output of machine learning model 908 may be used for further modeling 906.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The terms “controller”, “processor”, or “processing circuitry” may generally refer to any of the foregoing logic circuitry, alone or in combination with other logic circuitry, or any other equivalent circuitry. A control unit comprising hardware may also perform one or more of the techniques of this disclosure. Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), or electronically erasable programmable read only memory (EEPROM), or other computer readable media.

This disclosure includes the following non-limiting examples.

### Example 1

A medical system comprising: memory configured to store angiography data of a patient; and processing circuitry communicatively coupled to the memory, the processing circuitry being configured to: obtain the angiography data; determine, based on the angiography data, a location of a true lumen of a cardiac vessel; determine a location of a distal portion of a medical instrument; determine, based on the location of the true lumen and the location of the distal portion of the medical instrument, a navigation path for the distal portion of the medical instrument to the true lumen; and output a representation of the navigation path for display.

### Example 2

The medical system of example 1, wherein the processing circuitry is further configured to determine, based on the angiography data, a location of a lesion, wherein the processing circuitry determines the navigation path further based on the location of the lesion.

### Example 3

The medical system of example 2, wherein the lesion is a chronic total occlusion.

### Example 4

The medical system of any of examples 1-3, wherein as part of at least one of determining the location of the true lumen or determining the navigation path, the processing circuitry is configured to execute a machine learning model.

### Example 5

The medical system of example 4, wherein the machine learning model is trained on at least one of angiography data of chronic total occlusion (CTO) medical procedures, cine imaging data, annotations associated with medical instruments used during sub intimal CTO medical procedures, intravascular ultrasound data, or coronary computed tomography angiography data.

### Example 6

The medical system of example 4 or example 5, wherein as part of determining the navigation path, the processing circuitry is configured to: determine a first certainty measure associated with the determination of the location of true lumen; determine whether the first certainty measure satisfies a threshold; and based on the first certainty measure not satisfying the threshold, output a request to acquire additional angiography data at a different angiography angle than a current angiography angle.

### Example 7

The medical system of example 6, wherein the processing circuitry is further configured to: acquire the additional angiograph data; determine, based on the angiography data and the additional angiography data, a second estimated location of the true lumen; determine a second certainty measure associated with the determination of the second estimated location of the true lumen; determine whether the second certainty measure satisfies the threshold; and based on the second certainty measure satisfying the threshold, determining the navigation path.

### Example 8

The medical system of any of examples 1-7, wherein the representation of the navigation path comprises an instruction to navigate the distal portion of the medical instrument into the true lumen.

### Example 9

The medical system of any of examples 1-8, wherein the representation of the navigation path comprises a graphical representation of the navigation path.

### Example 10

The medical system of any of examples 1-9, wherein the processing circuitry is further configured to: obtain updated angiography data; determine, based on the updated angiography data, an updated location of the true lumen; determine an updated location of the distal portion of the medical instrument; determine, based on the updated location of the true lumen and the updated location of the distal portion of the medical instrument, whether the distal portion of the medical instrument is in the true lumen; and based on the distal portion of the medical instrument being in the true lumen, output an indication.

### Example 11

The medical system of example 10, wherein the processing circuitry is configured to output the indication for display.

### Example 12

The medical system of any of examples 1-11, wherein the navigation path comprises at least one of an angle of approach towards the true lumen, a direction of navigation towards the true lumen, or a distance from the medical instrument to the true lumen.

### Example 13

The medical system of any of examples 1-12, wherein the location of the true lumen comprises a location distal to a lesion.

### Example 14

A method comprising: obtaining, by processing circuitry, angiography data of a patient; determining, by the processing circuitry and based on the angiography data, a location of a true lumen of a cardiac vessel; determining, by the processing circuitry, a location of a distal portion of a medical instrument; determining, by the processing circuitry and based on the location of the true lumen and the location of the distal portion of the medical instrument, a navigation path for the distal portion of the medical instrument to the true lumen; and outputting, by the processing circuitry, a representation of the navigation path for display.

### Example 15

The method of example 14, further comprising determining, by the processing circuitry and based on the angiography data, a location of a lesion, determining the navigation path is further based on the location of the lesion.

### Example 16

The method of example 15, wherein the lesion is a chronic total occlusion.

### Example 17

The method of any of examples 14-16, wherein at least one of determining the location of the true lumen or determining the navigation path comprises executing, by the processing circuitry, a machine learning model.

### Example 18

The method of example 17, wherein the machine learning model is trained on at least one of angiography data of chronic total occlusion (CTO) medical procedures, cine imaging data, annotations associated with medical instruments used during sub intimal CTO medical procedures, intravascular ultrasound data, or coronary computed tomography angiography data.

### Example 19

The method of any of examples 14-18, wherein the location of the true lumen comprises a location distal to a lesion.

### Example 20

A non-transitory computer-readable storage medium storing instructions, which when executed cause processing circuitry to: obtain angiography data of a patient; determine, based on the angiography data, a location of a true lumen of a cardiac vessel; determine a location of a distal portion of a medical instrument; determine, based on the location of the true lumen and the location of the distal portion of the medical instrument, a navigation path for the distal portion of the medical instrument to the true lumen; and output a representation of the navigation path for display.

Various examples have been described. These and other examples are within the scope of the following claims.

1. A medical system comprising:
memory configured to store angiography data of a patient; and
processing circuitry communicatively coupled to the memory, the processing circuitry being configured to:
obtain the angiography data;
determine, based on the angiography data, a location of a true lumen of a cardiac vessel;
determine a location of a distal portion of a medical instrument;
determine, based on the location of the true lumen and the location of the distal portion of the medical instrument, a navigation path for the distal portion of the medical instrument to the true lumen; and
output a representation of the navigation path for display.

2. The medical system of claim 1, wherein the processing circuitry is further configured to determine, based on the angiography data, a location of a lesion, wherein the processing circuitry determines the navigation path further based on the location of the lesion.

3. The medical system of claim 2, wherein the lesion is a chronic total occlusion.

4. The medical system of claim 1, wherein as part of at least one of determining the location of the true lumen or determining the navigation path, the processing circuitry is configured to execute a machine learning model.

5. The medical system of claim 4, wherein the machine learning model is trained on at least one of angiography data of chronic total occlusion (CTO) medical procedures, cine imaging data, annotations associated with medical instruments used during sub intimal CTO medical procedures, intravascular ultrasound data, or coronary computed tomography angiography data.

6. The medical system of claim 4, wherein as part of determining the navigation path, the processing circuitry is configured to:
determine a first certainty measure associated with the determination of the location of true lumen;
determine whether the first certainty measure satisfies a threshold; and
based on the first certainty measure not satisfying the threshold, output a request to acquire additional angiography data at a different angiography angle than a current angiography angle.

7. The medical system of claim 6, wherein the processing circuitry is further configured to:
acquire the additional angiograph data;
determine, based on the angiography data and the additional angiography data, a second estimated location of the true lumen;
determine a second certainty measure associated with the determination of the second estimated location of the true lumen;
determine whether the second certainty measure satisfies the threshold; and
based on the second certainty measure satisfying the threshold, determining the navigation path.

8. The medical system of any claim 1, wherein the representation of the navigation path comprises an instruction to navigate the distal portion of the medical instrument into the true lumen.

9. The medical system of claim 1, wherein the representation of the navigation path comprises a graphical representation of the navigation path.

10. The medical system of claim 1, wherein the processing circuitry is further configured to:
obtain updated angiography data;
determine, based on the updated angiography data, an updated location of the true lumen;
determine an updated location of the distal portion of the medical instrument;
determine, based on the updated location of the true lumen and the updated location of the distal portion of the medical instrument, whether the distal portion of the medical instrument is in the true lumen; and
based on the distal portion of the medical instrument being in the true lumen, output an indication.

11. The medical system of claim 10, wherein the processing circuitry is configured to output the indication for display.

12. The medical system of claim 1, wherein the navigation path comprises at least one of an angle of approach towards the true lumen, a direction of navigation towards the true lumen, or a distance from the medical instrument to the true lumen.

13. The medical system of claim 1, wherein the location of the true lumen comprises a location distal to a lesion.

14. A method comprising:
obtaining, by processing circuitry, angiography data of a patient;
determining, by the processing circuitry and based on the angiography data, a location of a true lumen of a cardiac vessel;
determining, by the processing circuitry, a location of a distal portion of a medical instrument;
determining, by the processing circuitry and based on the location of the true lumen and the location of the distal portion of the medical instrument, a navigation path for the distal portion of the medical instrument to the true lumen; and
outputting, by the processing circuitry, a representation of the navigation path for display.

15. A non-transitory computer-readable storage medium storing instructions, which when executed cause processing circuitry to:
obtain angiography data of a patient;
determine, based on the angiography data, a location of a true lumen of a cardiac vessel;
determine a location of a distal portion of a medical instrument;
determine, based on the location of the true lumen and the location of the distal portion of the medical instrument, a navigation path for the distal portion of the medical instrument to the true lumen; and
output a representation of the navigation path for display.

16. The method of claim 14, further comprising determining, by the processing circuitry and based on the angiography data, a location of a lesion, determining the navigation path is further based on the location of the lesion.

17. The method of example 16, wherein the lesion is a chronic total occlusion.

18. The method of claim 14, wherein at least one of determining the location of the true lumen or determining the navigation path comprises executing, by the processing circuitry, a machine learning model.

19. The method of claim 18, wherein the machine learning model is trained on at least one of angiography data of chronic total occlusion (CTO) medical procedures, cine imaging data, annotations associated with medical instruments used during sub intimal CTO medical procedures, intravascular ultrasound data, or coronary computed tomography angiography data.

20. The method of claim 14, wherein the location of the true lumen comprises a location distal to a lesion.
